(19) United States
(12) Patent Application Publication
(10) Pub. No.: US 2006/0204595 A1
(43) Pub. Date: Sep. 14, 2006

(54) Antioxidant supplement composition for smokers and ex-smokers and method of producing thereof

(75) Inventors: Sun Ho Park, Saha-gu (KR); Ho Jae Lee, Haeundae-gu (KR)

Correspondence Address: DICKINSON WRIGHT PLLC, 1901 L. STREET NW, SUITE 800, WASHINGTON, DC, 20036, US

(73) Assignee: NOSMO CO., LTD., Busanjin-gu (KR)

(21) Appl. No.: 11/372,101

(22) Filed: Mar. 10, 2006

(30) Foreign Application Priority Data
Mar. 11, 2005 (KR) 10-2005-0020458

(51) Int. Cl. A61K 36/254; A61K 36/752; A61K 36/886; A61K 36/48; A61K 36/28; A61K 36/258

(52) U.S. Cl. 424/725; 424/728; 424/736; 424/757; 424/764; 424/744

(57) ABSTRACT

An antioxidant supplement composition includes a mixture including 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, 18-20% Arctium lappa, 30-200 mg vitamin-C, and 1,500-20,000 IU β-carotene.

### BACKGROUND OF THE INVENTION

1. Field of the Invention

The present invention generally relates to an antioxidant supplement composition suitable for smokers and ex-smokers, and a method of producing thereof, and more particularly, to an antioxidant supplement composition that can remove accumulated nicotine in smokers or ex-smokers and can supply antioxidant nutrients usually lacking in smokers or ex-smokers and a method of producing such an antioxidant supplement composition.

2. Background Art

Generally, antioxidant nutrients function to remove strong cell toxicity containing a reactive oxygen species produced during metabolic process.

Particularly, reactive oxygen species formed by cigarette smoking are associated with accelerating oxidative damage of proteins, lipids, and nucleic acids, and accelerating age-related diseases such as cancer, diabetes, joint inflammation and hardening of the arteries, and the like. Defense systems against the reactive oxygen species in the human body are classified into two groups. One is an enzyme-system such as superoxide dismutase, catalase, glutathion peroxidase, and the like. A second is small molecular weight antioxidants such as vitamin-A, vitamin-C, vitamin-E, caroteneoids, and the like.

When reactive oxygen species are accumulated in human bodies, large amounts of the antioxidant nutrients are consumed. Particularly, it is noted that the smokers may lack vitamin-C and carotenoids such as α-carotene, β-carotene, cryptoxantine, and the like (Wei W et al., 2001).

It is reported that the amount of vitamin-C in smokers is less than non-smokers by about 27%, and of ex-smokers by about 6% (Herbert J R et al., 1994). It is also noted that the amount of carotenoids contained in smokers are less than that in non-smokers by about 25% (Alberg A J et al., 2000 and Marangon K et al., 1998).

Many antioxidant compositions for removing reactive oxygen species have been proposed. Nevertheless, there have been no antioxidant compositions for supplementing limited antioxidants, for example smokers or ex-smokers.

### SUMMARY OF THE INVENTION

The present invention solves the foregoing problems and it is therefore the object of the present invention to provide an antioxidant supplement composition that includes medicinal herbs, vitamin-C and carotenoid, and can remove accumulated nicotine such as found in smokers or ex-smokers and can supply antioxidant nutrients that may be lacking in smokers or ex-smokers and to provide a method of producing such an antioxidant supplement composition.

According to an aspect of the invention to obtain the above objects, there is provided an antioxidant supplement composition suitable, for example, smokers and ex-smokers, including: a mixture of Glycyrrhiza uralensis, Crataegus pinnatifida, Ligusticum tenuissimum, Acathopanax sessiliflorus, Polygonum multiflorum, Eugenia aromaticum, Citrus reticulatae, Raphani seed, Platycodon grandiflorum, Aloe arborescens, Arctium lappa, vitamin-C, and carotenoid.

The mixture may include 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa.

The vitamin-C component may be about 30-200 mg and the carotenoid component may be 1,500-20,000 IU β-carotene.

According to another aspect of the present invention, there is provided a method of producing an antioxidant supplement composition suitable, for example, for smokers and ex-smokers including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; concentrating the extract with vacuum evaporation up to 10-15 brix; adding a small amount of bulking agents to the extract so that the final concentration of the extract becomes 15-20 brix; spray-drying the extract into a powder; mixing the powder in different ratios with 30-200 mg vitamin-C and 1,500-20,000 IU β-carotene; and forming the mixture in tablet types.

According to another aspect of the present invention, there is provided a method of producing an antioxidant supplement composition suitable for smokers and ex-smokers are provided including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; concentrating the extract with vacuum evaporation up to 10-15 brix; adding a small amount of bulking agents to the extract so that the final concentration of the extract becomes 15-20 brix; spray-drying the extract into a powder; mixing 1.0-5.0% of the powder with 30-200 mg vitamin-C, 1,500-20,000 IU β-carotene, gum base, sweeteners, and flavors; and forming the mixture in gum types.

According to another aspect of the present invention, there is provided a method of producing an antioxidant supplement composition, including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; concentrating the extract with vacuum evaporation up to 10-15 brix; adding a small amount of bulking agents to the extract so that the final concentration of the extract becomes 15-20 brix; spray-drying the extract into a powder; mixing 1.0-5.0% of the powder with 30-200 mg vitamin-C, 1,500-20,000 IU β-carotene, sweeteners, acidifiers, flavors, and water; and forming the mixture into an antioxidant supplement drink.

According to another aspect of the present invention, there is provided a method of producing an antioxidant supplement composition, including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; concentrating the extract with vacuum evaporation up to 10-15 brix; adding a small amount of bulking agents to the extract so that the final concentration of the extract becomes 15-20 brix; spray-drying the extract into a powder; mixing 1.0-5.0% of the powder with 30-200 mg vitamin-C, 1,500-20,000 IU β-carotene, powdered sugars, and flavors; and forming the mixture in candy types.

According to another aspect of the present invention, there is provided a method of producing an antioxidant supplement composition, including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; concentrating the extract with vacuum evaporation up to 55-65 brix; mixing 1.0-5.0% of the extract with 30-200 mg vitamin-C, 1,500-20,000 IU β-carotene, sweeteners, acidifiers, flavors, and water; and forming the mixture in antioxidant supplement drinks.

According to another aspect of the present invention, there is provided a method of producing an antioxidant supplement composition, including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; mixing 30-200 mg vitamin-C and 1,500-20,000 IU β-carotene with the extract; injecting the mixture into edible capsules to provide the antioxidant supplement composition in flexible capsule types.

According to another aspect of the present invention, there is provided a method of producing an antioxidant supplement composition, including: preparing a mixture comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa; extracting the medicinal herbs with 80% alcohol which is 7 times of a mixture and refluxing at 85° C. for 4 hours; mixing 1.0-5.0% of the extract with 30-200 mg vitamin-C, 1,500-20,000 IU β-carotene, gum base, sweeteners, and flavors; and forming the mixture in gum types.

### DETAILED DESCRIPTION OF THE EMBODIMENT

According to the present invention, an antioxidant supplement composition can remove accumulated nicotine such as in smokers or ex-smokers and can supply antioxidant nutrients that are usually lacking in their bodies.

The following detailed description will present a preferred embodiment of the invention in reference to the accompanying tables.

According to the present invention, twenty-two medicinal herbs were selected based on efficacy relating to the bronchus and lungs as documented in the Oriental medicine books (Donguibogam, famous Korean medical book by Heo Jun(1546˜1615)) and otherwise known in the art. They are shown in table 1.

TABLE 1

| No. | Herbs |
|---|---|
| 1 | Glycyrrhiza uralensis Fisch |
| 2 | Ostericum koreanum Kitagawa |
| 3 | Zingiber officinale Roscoe |
| 4 | Ligusticum tenuissimum Nakai |
| 5 | Platycodon grandiflorum |
| 6 | Raphani seed |
| 7 | Liriope platyphylla Wang et Tang |
| 8 | Angelica dahuricae |
| 9 | Sophora subprostrata Chun et T Chun |
| 10 | Crataegus pinnatifida |
| 11 | Saururus chinensis (Lour Baill) |
| 12 | Cimicifuga heracleifolia Komarov |
| 13 | Aloe arborescens |
| 14 | Acathopanax sessiliflorus Seemen |
| 15 | Panax ginseng C. A. Meyer |
| 16 | Anthriscus sylvestris Hoffmann |
| 17 | Eugenia aromaticum |
| 18 | Citrus reticulatae |
| 19 | Alisma canaliculatum All. Br. et Bouche |
| 20 | Polygonum multiflorum Thunberg |
| 21 | Astrgalus membranaceus Bunge |
| 22 | Arctium lappa |

A test sample was prepared for measuring the antioxidant activity and nicotine degradation activity using the selected medicinal herbs.

The selected medicinal herbs were cut into small pieces, and log of each sample were put into 500 mL 80% methanol, shaken for 24 hours, and filtered with Toyo No. 1 filter paper. The filtered extracts were concentrated and dried using a rotary vacuum evaporator (Eyela NEIS, Japan).

The sample was re-solubilized to a final volume of 10 ml with 80% methanol. These samples were next stored at −20° C. in the dark and thawed just before use.

1) Antioxidant Activity Measurement

The antioxidant activity of the sample was measured by DPPH(α,α-diphenyl-β-picrylhydrazyl) free radical scavenging method (Williams et al., 1995). The absorbance at 517 nm was monitored in the presence of different concentrations of the sample extracts. The control, 80% methanol instead of the sample solution, was monitored to determine the absorbance of DPPH before interacting with the sample extracts. The antioxidant activity was expressed as IC₅₀(μg/mL), the amount of me-dicinal herbs necessary to decrease 50% of the initial DPPH absorbance.

The result on DPPH free radical scavenging activities of the selected 22-medicinal herbs are shown in Table 2.

TABLE 2

| | | IC₅₀ | |
|---|---|---|---|
| No | Medicinal Herbs | (μg/mL) | NDA |
| 1 | Glycyrrhiza uralensis | 69^{cd} | 1.10^{c} |
| 2 | Ostericum koreanum | 86^{e} | 1.60^{k} |
| 3 | Zingiber officinale | 84^{de} | 1.50^{j} |
| 4 | Ligusticum tenuissimum | 369^{jk} | 1.04^{b} |
| 5 | Platycodon grandiflorum | 921^{m} | 1.13^{d} |
| 6 | Raphani seed | 346ⁱ | 1.01^{a} |
| 7 | Liriope platyphylla | 2880^{o} | 1.12^{d} |
| 8 | Angelica dahuricae | 355^{ij} | 1.20^{e} |
| 9 | Sophora subprostrata | 308^{h} | 1.01^{a} |
| 10 | Crataegus pinnatifida | 51^{b} | 1.75^{l} |
| 11 | Saururus chinensis | 54^{bc} | 1.24^{g} |
| 12 | Cimicifuga heracleifolia | 64^{bc} | 1.39ⁱ |
| 13 | Aloe arborescens | 380^{k} | 1.05^{b} |
| 14 | Acathopanax sessiliflorus | 54^{bc} | 1.23^{fg} |
| 15 | Panax ginseng | 3270^{p} | 1.30^{h} |
| 16 | Anthriscus sylvestris | 731^{l} | 1.60^{k} |
| 17 | Eugenia aromaticum | 30^{a} | 1.22^{f} |
| 18 | Citrus reticulatae | 125^{g} | 1.23^{fg} |
| 19 | Alisma canaliculatum | 104^{f} | 1.10^{c} |
| 20 | Polygonum multiflorum | 2280ⁿ | 1.28^{h} |
| 21 | Astrgalus membranaceus | 744^{l} | 1.81^{m} |
| 22 | Arctium lappa | 32^{a} | 1.79^{m} |

As shown in Table 2, the IC₅₀ values of the Eugenia aromaticum (30 μg/mL), Arctium lappa (32 μg/mL), Crataegus pinnatifida (51 μg/mL), Acathopanax sessiliflorus (54 μg/mL), Saururus chinensis (53 μg/mL), Cimicifuga heracleifolia (64 μg/mL), Glycyrrhiza uralensis (69 μg/mL), Ostericum koreanum (104 μg/mL), Zingiber officinale (84 μg/mL), Alisma canaliculatum (104 μg/mL), and Citrus reticulatae (125 μg/mL) were less than 150 μg/mL. Particularly, the Eugenia aromaticum (30 μg/mL) and Arctium lappa (32 μg/mL) have the highest free radical scavenging activity.

2) Nicotine Degradation Activity (NDA) Measurement

The NDA was measured using cultured PLC/PRF4 cell. The pre-cultured cells were washed with phosphate buffered saline (PBS) and cultured for 24 hours in a 3 mL media containing 10 μL nicotine stock solu-tion. After 10 μL of each medicinal herb sample extract were added to the culture solution, and further cultured for 1 hour, and concentration of cotinine converted from nicotine was measured. The cotinine concentration was measured by the modified method of Barlow et al. (1987) at 490 nm.

NDA was calculated by the absorbance ratio of 1 hour compared to 0 hour's after sample treatment.

The NDA values of medicinal herbs determined using PLC/ PRF5 cells are shown in Table 2.

There were significant differences in NDA among some medicinal herbs (p<0.05). The highest and lowest NDA were 1.81 and 1.01 absorbance ratio of treatment/control at 490 nm respectively.

Among the 14 medicinal herbs, 9 medicinal herbs showing a higher NDA of over 1.20 had high anti-oxidant activity with IC₅₀ values of less than 150 μg/mL.

Nevertheless, some medicinal herbs including the Astrgalus membranaceus, Panax ginseng, Anthriscus sylvestris, and Ligusticum tenuissimum showed high NDA values and low antioxidant activities. Ostericum koreanum, Zingiber officinale, Ligusticum tenuissimum, Platycodon grandiflorum, Raphani seed, Sophora subprostrata, Crataegus pinnatifida, Arctium lappa, Cimicifuga heracleifolia, Aloe arborescens, and Citrus reticulatae showed a relatively high coefficient of determination (R²=0.840) between antioxidant activities and NDA values, while the others showed low correlation (R²=0.271).

These results indicate the antioxidant activity of the medicinal herbs may be closely related to the NDA. That is, some selected medicinal herbs with high antioxidant activity and/or NDA can be used as preferable materials for producing an antioxidant supplement composition suitable for smokers and ex-smokers.

Examples of the present invention were prepared using the medicinal herbs chosen based on the high antioxidant activity and NDA.

That is, 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Platycodon grandiflorum, 21-26% Aloe arborescens, and 18-20% Arctium lappa were mixed each other.

The herb mixtures were extracted with 80% alcohol which is 7 times of a mixture and refluxed at 85° C. for 4 hours. The extract was concentrated with vacuum evaporation up to 10-15 brix. Then, a small amount of bulking agents was added to the extract so that the final concentration of the extract becomes 15-20 brix. Then, the extract was spray dried into powder. The powder of the medicinal herb extracts was mixed in different ratios with additional 100 mg vitamin-C and 3,000 IU β-carotene in ratios as shown in the table 3.

TABLE 3

| Medicinal Herbs | Ex 1 | Ex 2 | Ex 3 | Ex 4 | Ex 5 | Ex 6 | Ex 7 | Ex 8 | Ex 9 |
|---|---|---|---|---|---|---|---|---|---|
| Glycyrrhiza uralensis | 20 | 24 | 26 | 27 | 20 | 20 | 20 | 22 | 22 |
| Ligusticum tenuissimum | 3 | 1 | 3 | 1 | 1 | 1 | 2 | 3 | 3 |
| Platycodon grandiflorum | 6 | 7 | 5 | 5 | 10 | 7 | 6 | 6 | 6 |
| Raphani seed | 6 | 4 | 4 | 3 | 3 | 5 | 3 | 5 | 4 |
| Crataegus pinnatifida | 4 | 3 | 6 | 5 | 2 | 6 | 5 | 3 | 3 |
| Aloe arborescens | 23 | 23 | 21 | 21 | 26 | 22 | 24 | 24 | 23 |
| Acathopanax sessiliflorus | 3 | 3 | 2 | 2 | 4 | 4 | 3 | 2 | 2 |
| Eugenia aromaticum | 3 | 3 | 5 | 5 | 6 | 6 | 4 | 4 | 3 |
| Citrus reticulatae | 9 | 10 | 8 | 10 | 8 | 9 | 8 | 9 | 10 |
| Polygonum multiflorum | 5 | 3 | 2 | 2 | 2 | 2 | 5 | 4 | 5 |
| Arctium lappa | 18 | 19 | 18 | 19 | 18 | 18 | 20 | 18 | 19 |
| Vitamin-C | 100 | 100 | 100 | 100 | 100 | 100 | 100 | 100 | 100 |
| β -carotene | 3000 | 3000 | 3000 | 3000 | 3000 | 3000 | 3000 | 3000 | 3000 |

The antioxidant activities and the NDAs of the examples are shown in Table 4. The IC₅₀ value and NDA were in a range of 99-112 μg/mL and 1.28-1.38, respectively. This indicates some examples have a relatively high antioxidant activity and a relatively high NDA.

TABLE 4

| | Test Items | | | | | | | | |
|---|---|---|---|---|---|---|---|---|---|
| | Ex 1 | Ex 2 | Ex 3 | Ex 4 | Ex 5 | Ex 6 | Ex 7 | Ex 8 | Ex 9 |
| IC₅₀(μg/mL) | 99 | 109 | 108 | 110 | 102 | 112 | 106 | 105 | 109 |
| NDA | 1.38 | 1.30 | 1.33 | 1.33 | 1.36 | 1.28 | 1.37 | 1.36 | 1.29 |

Example 1, the antioxidant supplement composition having the highest antioxidant activity and NDA, was selected and modified mixture ratios based on Example 1 were prepared as shown in Table 5.

TABLE 5

| Herbs | Ex 10 | Ex 11 | Ex 12 | Ex 13 |
|---|---|---|---|---|
| Glycyrrhiza uralensis | 20 | 20 | 20 | 20 |
| Ligusticum tenuissimum | 3 | 3 | 3 | 3 |
| Platycodon grandiflorum | 6 | 6 | 6 | 6 |
| Raphani seed | 6 | 6 | 6 | 6 |
| Crataegus pinnatifida | 4 | 4 | 4 | 4 |
| Aloe arborescens | 23 | 23 | 23 | 23 |
| Acathopanax sessiliflorus | 3 | 3 | 3 | 3 |
| Eugenia aromaticum | 3 | 3 | 3 | 3 |
| Citrus reticulatae | 9 | 9 | 9 | 9 |
| Polygonum multiflorum | 5 | 5 | 5 | 5 |
| Arctium lappa | 18 | 18 | 18 | 18 |
| Vitamin-C | 30 | 60 | 120 | 200 |
| β -carotene | 1500 | 6000 | 12000 | 20000 |

The antioxidant activities and the NDAs of EX1, EX10, EX11, EX12, and EX13 were measured and shown in Table 6. The IC₅₀ value was in a range of 91-123 μg/mL and the NDA was in a range of 1.32-1.41. This shows that these examples have relatively high antioxidant activity and high NDA.

TABLE 6

| | Test Items | | | | |
|---|---|---|---|---|---|
| | Ex 1 | Ex 10 | Ex 11 | Ex 12 | Ex 13 |
| IC₅₀(μg/mL) | 99 | 123 | 106 | 96 | 91 |
| NDA | 1.38 | 1.32 | 1.33 | 1.41 | 1.36 |

In vivo experiments were performed using male ICR rats. In the experiments, the preventing effect of lipid peroxidation of the lung cell, which may be caused by the active oxygen species generated by smoking was measured to evaluate the efficacy of the antioxidant supplement compositions of on reducing the oxidative damages induced from smoking,

Rats were randomly divided into 6 groups: a control and five groups of example compositions-treated. The number of rats of each group was 10 and the rats of each group freely had normal solid feeds (Samyangsa Co. Ltd.) for 8 weeks. Additionally, they were exposed to cigarette smoke equivalent to 10 cigarettes per day by a smoke supplying apparatus. In the example compositions-treated groups, each example composition (10 mg per 100 g body weight) was given in drinking water. The control rats were given only normal drinking water. After 8 weeks of each treatment, lung cells were taken from the rats of each group and the amount of lipid peroxidized material was measured and represented as free malondialdehyde (MDA). MDA is defined as an MDA equivalent (%), which was determined through following procedures: add 2 mL trichloroacetate-HCl (TBA) solution to 1 mL of sample, heat the sample with TBA in boiling water for 15 minutes, cool down the sample, separate upper liquid layer after centrifugation, measured the absorbance of the sample at 535 nm, and calculate percentage relative to the values of control groups.

TABLE 7

| | Ex 1 | Ex 10 | Ex 11 | Ex 12 | Ex 13 |
|---|---|---|---|---|---|
| MDA | 28.3 ± 1.3 | 35.5 ± 4.2 | 31.1 ± 9.6 | 26.7 ± 2.9 | 27.5 ± 6.4 |
| equivalent | | | | | |
| (%) | | | | | |

In Table 7, the antioxidant activities in an animal body with respect to the antioxidant supplement compositions are represented as the MDA equivalents (%). Table 7 shows the antioxidant supplement compositions of the examples can prevent the lipid peroxidation induced from smoking.

In addition, Table 7 shows that the composition of the example 12 is the most effective in the antioxidant activity.

The method of producing the antioxidant supplement composition of the example 12 are described below.

The medicinal herbs were prepared according to a ratio of the example 12 and mixed. The mixture was extracted with 80% alcohol which is 7 times of a mixture and refluxed at 85° C. for 4 hours. Then, the extract was concentrated with vacuum evaporation up to 10-15 brix. Then, a small amount of bulking agent was added to the extract so that the final concentration of the extract becomes 15-20 brix. Then, the extract was spray dried into powder (named as NPL-X). The NPL-X was mixed in different ratios with 200 mg vitamin-C and 20,000 IU β-carotene. This mixture may be formed in tablet types.

Alternatively, 1.0-5.0% of NPL-X was mixed with 30 mg vitamin-C and 1,500 IU β-carotene. This mixture was mixed with gum base, known in the art sweeteners, and flavors, thereby providing an antioxidant supplement gums.

Alternatively, 1.0-5.0% of NPL-X was mixed with 150 mg vitamin-C and 1,500 IU β-carotene. This mixture was mixed with sweeteners, acidifiers, flavors, and water, thereby providing an antioxidant supplement drinks.

Alternatively, 1.0-5.0% of NPL-X was mixed with 200 mg vitamin-C and 1,500 IU β-carotene. This mixture was mixed with powdered sugars, and flavors, thereby providing an antioxidant supplement candies.

Alternatively, the medicinal herbs were prepared according to a ratio of the example 12 and mixed. The mixture was extracted with 80% alcohol which is 7 times of a mixture and refluxed at 85° C. for 4 hours. Then, the extract was concentrated with vacuum evaporation up to 55-65 brix. One to five percentage of this concentration (named as NPL-C) was mixed with 150 mg vitamin-C and 3,000 IU β-carotene. This mixture was mixed with sweeteners, acidifiers, flavors, and water, thereby providing an antioxidant supplement drink.

Alternatively, 1.0-5.0% of NPL-C was mixed with 200 mg vitamin-C and 20,000 IU β-carotene. This mixture was injected into edible capsules, thereby providing antioxidant supplement capsules.

Further alternatively, 1.0-5.0% of NPL-C was mixed with 30 mg vitamin-C and 1,500 IU β-carotene. This mixture was mixed with gum base, sweeteners, and flavors, thereby providing an antioxidant supplement gums.

It will be apparent to those skilled in the art that various modifications and variations can be made in the present invention. Thus, it is intended that the present invention covers the modifications and variations of this invention provided they come within the scope of the appended claims and their equivalents.

As described above, an antioxidant supplement composition according to the present invention can remove accumulated nicotine such as in smokers or ex-smokers and can supply antioxidant nutrients usually lacking in their bodies.

What is claimed is:

1. An antioxidant supplement composition, comprising:
a mixture of an extract from Glycyrrhiza uralensis, Crataegus pinnatifida, Ligusticum tenuissimum, Acathopanax sessiliflorus, Polygonum multiflorum, Eugenia aromaticum, Citrus reticulatae, Raphani seed, Pltycodon grandiflorum, Aloe arborescens, and Arctium lappa, vitamin-C, and carotenoid.

2. The antioxidant supplement composition of claim 1, wherein the extract comprises 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Pltycodon grandiflorum, 21-26% Aloe arborescens, and 18-20% Arctium lappa.

3. The antioxidant supplement composition of claim 1, wherein the vitamine-C is about 30-200 mg and the carotinoid is about 1,500-20,000 IU β-carotene.

4. A method of producing an antioxidant supplement composition for smokers and ex-smokers, comprising:
preparing a mixture of medical herbs comprising 20-27% Glycyrrhiza uralensis, 2-6% Crataegus pinnatifida, 1-3% Ligusticum tenuissimum, 2-4% Acathopanax sessiliflorus, 2-5% Polygonum multiflorum, 3-6% Eugenia aromaticum, 8-10% Citrus reticulatae, 3-6% Raphani seed, 5-10% Pltycodon grandiflorum, 21-26% Aloe arborescens, and 18-20% Arctium lappa;
extracting the medical herbs with 80% alcohol which is 7 times of a mixture and refluxed at 85° C. for 4 hours;
concentrating the extract up to 10-15 brix; and
adding a small amount of a bulking agent to the extract so that the final concentration of the extract becomes powder having 15-20 brix.

5. The method of claim 4, further comprising concentrating the extract into with vacuum evaporation up to 55-65 brix; and
mixing 1.0-5.0% extract of 55-65 brix with 30-200 mg vitamin-C and 1,500-20,000 IU β-carotene, sweeteners, acidifier, and flavors to be formed into beverage types.

6. The method of claim 4, wherein the antioxidant supplement composition is formed in a gum type including 1.0-5.0% powder, 30 mg vitamin-C, 1,500 IU β-carotene, gum base, sweeteners, and flavors.

7. The method of claim 4, wherein the antioxidant supplement composition is formed in a candy type including 1.0-5.0% powder, 200 mg vitamin-C, 1,500 IU β-carotene, powder sweeteners and powder flavors.

8. The method of claim 4, further comprising concentrating the extract into with vacuum evaporation up to 55-65 brix, wherein a mixture is prepared by mixing 1.0-5.0% extract of 55-65 brix with 200 mg vitamin-C and 20,000 β-carotene and the mixture is injected in a edible film to be a flexible capsule type.

9. The method of claim 4, further comprising concentrating the extract into with vacuum evaporation up to 55-65 brix, wherein the the antioxidant supplement composition is formed in a gum type including 1.0-5.0% extract of 55-65 brix, 30 mg vitamin-C, 1,500 IU β-carotene, gum base, sweeteners, and flavors.

10. The method of claim 4, further comprising mixing the powder in different ratios with vitamin-C 30˜200 mg and 1,500˜20,000 IU β-carotene and forming the mixture into a tablet type.

11. An antioxidant supplement composition wherein, the antioxidant supplement composition being formed in a beverage type includes 1.0-5.0% powder obtained in the method of claim 4, 150 mg vitamin-C, 1,500 IU β-carotene, sweeteners, acidifier and flavors.
